(19) United States
(12) Patent Application Publication
(10) Pub. No.: US 2008/0280357 A1
(43) Pub. Date: Nov. 13, 2008

(54) Cryopreservation of Hepatocytes

(76) Inventors: Lubomir Arseniev, Hannover (DE); Krassimira Alexandrova, Hannover (DE); Marc Barthold, Hannover (DE); Sabine Kafert-Kasting, Hannover (DE); Britta Laube, Hannover (DE)

Correspondence Address: OSTROLENK FABER GERB & SOFFEN, 1180 AVENUE OF THE AMERICAS, NEW YORK, NY, 100368403, US

(21) Appl. No.: 12/094,335

(22) PCT Filed: Nov. 3, 2006
(86) PCT No.: PCT/EP06/10549

§ 371 Date: Jun. 11, 2008

(30) Foreign Application Priority Data
Nov. 25, 2005 (DE) 10 2005 057 106.9

(51) Int. Cl. C12N 5/06

(52) U.S. Cl. 435/370; 435/374; 435/325

(57) ABSTRACT

The invention relates to processes for the preparation of liver cells for cryopreservation, processes for cryopreservation of isolated liver cells and processes for the preparation of a culture of cryopreserved isolated liver cells.

The present application relates to the technical field of cryopreservation and specifically to processes for the preparation of liver cells for cryopreservation, processes for cryopreservation of isolated liver cells and processes for the preparation of a sandwich culture of cryopreserved isolated liver cells.

### PRIOR ART

Liver cells isolated from the tissue complex, specifically hepatocytes, are employed especially in the form of primary cell cultures for testing the physiological action of drug candidates. Freshly prepared primary hepatocytes from humans, especially, represent the “gold standard” for determining active substance candidates in in vitro test series or carrying out investigations on the metabolism of the active substances or for enzyme induction. Disadvantageously, freshly isolated human hepatocytes are not available regularly and at any time. There is therefore the need for processes by which isolated hepatocytes can be stored for a certain time. The physiological function of the cells, that is especially their metabolic or enzymatic competence, should be retained as completely as possible here.

As is known, hepatocytes are stored in cryopreserved form. Generally, hepatocytes are cryopreserved in suspension. In addition to the hepatocytes, the suspension additionally contains a freezing medium which is intended to prevent damage to the cells by freezing and thawing. For storage, the suspension is usually frozen at temperatures below −80° C. For use after storage, the frozen cell suspension is thawed, and the cells are plated out on culture plates or in culture vessels. For reculturing of the thawed cells, the culture vessels are generally coated with matrix material, to which the cells can adhere. Successful adhesion is essential for the reculturing and the subsequent investigation. Usually, however, only a small proportion of the originally frozen cells can be preserved in the vital state and recultured. The disadvantage of this procedure consists especially in the fact that it cannot be foreseen whether or to what proportion the thawed cells from the suspension adhere to the culture plate. The proportion of adherent cells in the batch is dependent on the individual batch. Regularly, the thawed cryopreserved cells only adhere adequately to the culture plate in a small number of the batches.

A further known process for cryopreservation consists in plating out freshly isolated cells in culture vessels in order to freeze the plated out cells subsequently together with the culture vessels. Here too, the culture vessels are coated before plating out with a matrix to which the cells can adhere. As is known, collagen gels are preferably employed. In a known process, hepatocytes are inoculated onto a culture plates coated with collagen type I and allowed to adhere to the collagen matrix for about 4 hours. The resulting monolayer culture (single-layer cell culture) is then cultured for approximately 20 hours. Subsequently, non-adherent cells are washed off. After a further 6 hours, the culture is covered with a layer of freezing medium, cooled to −70° C. and stored (Watts and Grant, 1998; Human & Experimental Toxicology 15:30-37). For subsequent use, the monolayer cultures are thawed and recultured. The thawed cultures, however, contain a high proportion of nonadherent and non-vital cells and cell debris. Although usually large parts of the matrix are covered with cells, a confluent, continuous union of cells is not established.

In another known process, hepatocytes are frozen in sandwich configuration, that is in a double gel arrangement. For this, a hepatocyte suspension is first inoculated on cell culture plates coated with collagen gel. After culturing the cells for 24 hours, a second layer of collagen gel is poured onto the inoculated cells. Subsequently, the sandwich culture thus obtained is frozen at −70° C. in freezing medium and stored (Koebe et al., 1990; Cryobiology 27:576-584).

By the immobilization of the liver cells on a matrix or between two matrices before freezing, the cells are stabilized mechanically and the survival rate is thereby increased. On account of the fact that the cells can adhere in the freshly isolated, highly vital stage, the adhesion rate is markedly increased compared to the adhesion of cryopreserved cells frozen and thawed in suspension. Despite this, here too large proportions of nonadherent or nonvital cells occur after thawing. The ideal state, a confluent culture of vital hepatocytes, is not achieved. Moreover, it is seen that the cells frozen in suspension generally lose their metabolic competence within a few hours after thawing. They are then unsuitable for a large number of in vitro tests.

Further, cells isolated from human organs are usually less robust than cells removed from animal models. This is especially to be attributed to the fact that human cells from donor organs can generally be obtained under less favorable conditions than cells from animals which are usually expressly raised for this purpose and then euthanized (for example rats, mice or pigs). Therefore human cells require by far gentler culturing conditions. Up to now, no cryopreservation processes are known with which human hepatocytes can be successfully cryopreserved in order thus to be able subsequently to carry out in vitro studies over a longer period of time.

There is therefore the need for improved processes for cryopreservation of liver cells which make possible gentle cryopreservation, that is especially gentle thawing with a high survival rate. An improved cryopreservation process should especially be suitable for successfully cryopreserving isolated human hepatocytes. It should be guaranteed here that the thawed cells can be recultured to a large proportion on the culturing matrix, if possible as a confluent monolayer (continuous single layer). The process should further be suitable for being able to prepare improved sandwich cultures of isolated liver cells which contain a high proportion of vital cells. Further, it should be guaranteed that the recultured cells thawed after cryopreservation maintain their metabolic and/or enzymatic competence over as long a period of time as possible, such that they can be employed for a large number of in vitro tests.

### OBJECTIVE

The technical problem underlying the present invention accordingly consists in the provision of improved processes for the cryopreservation of isolated liver cells, in particular human liver cells, such that cryopreserved liver cells can finally be recultured as an improved sandwich culture.

The underlying technical problem is solved by a process as set forth in patent claim 1, in particular a process for the preparation of liver cells for cryopreservation, which contains the following steps:

In step (a), a matrix, in particular a collagen matrix, preferably a collagen gel, is prepared. The matrix is preferably introduced into a cell culture vessel, for example a 6-well plate. The culture vessel is preferably coated with matrix material.

In step (b), isolated liver cells, in particular isolated from tissue, are prepared.

In the subsequent step (c), the isolated liver cells are inoculated on the matrix. The density of the liver cells on the matrix here is from 2 to 4×10³ cells per mm² of matrix surface. The preferred cell density is from 2.6 to 3.2×10³ mm⁻². That is, in a culture vessel having a base area of 9.6 cm², for example the bottom surface of a well in a 6-well plate, the number of inoculated cells is from 2.5 to 3×10⁶ per well.

In the further, preferably immediately subsequent, step (d), the cells are allowed to rest on the matrix (resting phase, adhesion phase). For this, the matrix coated with cells is allowed to rest for a period of time of 10 to 180 minutes, preferably from 30 to 90 minutes, particularly preferably for approximately 1 hour, so that the cells inoculated on the matrix can adhere to the matrix. Resting preferably takes place in the culture cabinet (incubator), in particular under standard conditions at a temperature of 37° C., a proportion of 5% of CO₂ and at 95% relative humidity. Successful adhesion can additionally be verified by microscopic checking.

After the resting in step (d), the cells not adhering to the matrix in step (e) are washed off, in particular carefully, of the matrix coated with cells (washing step). Washing off is preferably carried out by covering the matrix coated with cells with a layer of culture medium, and subsequently aspirating the liquid supernatant of culture medium and nonadherent cells. This step is preferably repeated at least once.

In a further step (f) the washed-off matrix coated with cells is again allowed to rest (second resting phase). Resting takes place for a period of time of at most 180 minutes, particularly of 30 to 180 minutes, particularly preferably for approximately 1 hour. Resting preferably takes place in the culture cabinet, in particular under standard conditions, at a temperature of 37° C., a proportion of 5% of CO₂ and at 95% relative humidity.

After the second resting phase, the matrix coated with cells is frozen in a freezing medium in step (g) (freezing step). Before freezing and after the second resting phase, the matrix coated with cells is preferably washed off again, especially in order to remove residual, nonadherent cells (washing step). The procedure preferably corresponds to step (e).

The process according to the invention also proposes to inoculate isolated liver cells on a matrix in a certain density and, after a certain sequence of resting phases and washing off, to freeze nonadherent cells in freezing medium. Thus a matrix coated with cells, in particular a collagen matrix, to which the cells adhere and are particularly present in a monolayer, is frozen. By means of the measures according to the invention, surprisingly a culture of intact cells is obtained, which can be frozen, that is cryopreserved, particularly well. It is emphasized here that the cultures prepared and cryopreserved according to the invention are particularly viable (vital) after thawing and contain a high number of cells adherent to the matrix. The thawed cells can advantageously be recultured here in a confluent monolayer.

The process according to the invention is surprisingly particularly gentle to the cells and therefore also suitable for less robust cells, such as the hepatocytes isolated from human organs. Especially, it appears that the cells prepared and cryopreserved according to the invention, if they are covered with a layer of a second matrix after thawing, retain their full metabolic activity and/or metabolic competence for a number of days, particularly more than 3 days.

In a preferred embodiment of the process according to the invention, the freezing in step (g) is carried out with freezing medium which is added in an amount of approximately 0.5 ml per mm² of base area of the culture vessel. For this, the matrix coated with cells is preferably covered with a layer of the freezing medium. Preferably, the freezing medium contains 10% of fetal calf serum (FCS) and 10% of dimethyl sulfoxide (DMSO).

In a particularly preferred embodiment, in step (g) controlled cooling, that is freezing, takes place after addition of a freezing medium, preferably to temperatures of −80° C. or less. Preferably, cooling rates of −0.5 to −20° C. per minute are employed for the cooling. In a preferred variant, the phase transition is compensated here; this is preferably carried out by brief heating at heating rates of preferably from +1 to +3° C./minute.

After the preparation and freezing according to the invention of the isolated liver cells on a matrix, the matrix coated with cells is stored in the frozen state in the course of cryopreservation. Preferably, the temperature during storage is −80° C. or less, particularly preferably −150° C. or less. Expediently, storage takes place in a freezer or in the vapor phase of liquid air, or liquid nitrogen. Of course, all other known processes for low temperature storage of cells are also suitable. The person skilled in the art will choose the storage processes according to his field of application and according to their suitability.

The present invention accordingly also relates to a process for the cryopreservation of isolated liver cells, which contains the steps (a) to (g) characterized above, where in a further step (h) the frozen matrix coated with cells is stored for an indeterminate period of time, which is chosen depending on the field of application and intended use.

In a subsequent further step (i), depending on the field of application and intended use, preferably immediately before use or at another suitable point in time, the frozen matrix coated with cells is thawed again. This is preferably carried out by covering the frozen matrix coated with cells with a layer of warm culture medium. Preferably, the frozen matrix coated with cells is present in a culture vessel, for example a 6-well plate. Preferably, the culture vessel is removed from the freezer or the nitrogen tank and, in particular immediately thereafter, incubated for approximately 5 minutes under standard conditions (37° C., 5% CO₂, 95% relative humidity) in the culture cabinet. Preferably, a thawing medium (medium 1) is then pipetted, preferably slowly and dropwise, onto the matrix coated with cells. As a reference point, about 1 ml of medium 1 is added to an area of approximately 9.6 cm² (6-well plate). The temperature of the medium is preferably approximately 37° C. The process is repeated for each cell culture vessel. As a reference point, at most three 6-well plates are employed, such that the matrix coated with cells is covered with a layer of warm medium in at most 18 wells. Subsequently, each cell culture vessel or well is preferably coated in the same manner, preferably slowly and dropwise, with a layer of the same amount of medium 1 (1 ml per 9.6 cm²). In a preferred embodiment, medium 1 is a serum-containing thawing medium, which preferably contains 10% of fetal calf serum (FCS).

In a further process step (j), especially nonadherent cells or detached, nonvital cells are thus washed off from the matrix coated with cells, such that a matrix free of nonadherent cells is obtained. For this, the supernatant above the matrix coated with cells obtained by the addition of medium 1 in step (i) is at first aspirated as completely as possible. The supernatant essentially contains thawed, nonvital and nonadherent cells. In a particularly preferred embodiment, after this a further medium, that is medium 2, is added, preferably dropwise, to the aspirated matrix coated with cells. Medium 2 preferably has a temperature of approximately 37° C. In the case of the addition of medium 2, the two-step procedure presented in step (i) is preferably chosen; initially the first half of the medium is distributed in all cell culture vessels in each case and subsequently the second half is correspondingly distributed. The amounts of medium 2 added correspond to the amounts of medium 1 in step (i). Medium 2 preferably has a composition differing from the thawing medium (medium 1). Medium 2 is preferably serum-containing and preferably contains 10% of FCS.

In a particularly preferred embodiment, the matrices coated with cells and covered with a layer of medium 2 are then incubated under standard conditions (resting phase, incubation) in the culture cabinet for approximately 30 minutes.

For the removal of nonadherent and nonvital cells, the supernatant above the matrix coated with cells is then aspirated from the cell culture vessel as completely as possible. A matrix coated with cells which is essentially freed from nonadherent and nonvital cells is thereby obtained.

In a further step (k), the thawed, washed matrix coated with cells is covered with a layer of a second matrix, preferably of a collagen matrix, particularly preferably a collagen gel, or the gel is poured on. Preferably, the gel preferably poured on hardens within a few minutes to 1 hour. In a preferred embodiment, the composition of the first, lower matrix and of the second, upper matrix applied after thawing is essentially the same, preferably identical. Preferably, the second upper matrix is poured onto the monolayer of thawed liver cells adhering to the lower matrix as a gel. According to the invention, a sandwich culture is thus obtained, in which isolated liver cells are embedded between two matrices, in particular two collagen gels.

In a final step (l), the cells embedded between the matrices are recultured and, depending on the field of application and intended use, used as intended, that is preferably in in vitro tests, immediately or after a suitably chosen culturing period.

The thawed and recultured isolated liver cells prepared and cryopreserved according to the invention have a particularly high vitality rate. They show their metabolic competence for a particularly long time after thawing. Advantageously, less robust hepatocytes isolated from human organs can especially also be cryopreserved and successfully thawed by the procedure according to the invention, so that they can subsequently be used over a large period of time in appropriate in vitro tests. Surprisingly, a cell culture is obtained in which the cells can essentially be recultured as a confluent monolayer and in which the proportion of nonvital and/or nonadherent cells is very low.

The invention accordingly also relates to a process for the preparation of a sandwich culture of cryopreserved isolated liver cells, where at least the steps (a) to (l) of the process according to the invention are carried out and a sandwich culture of cells embedded between an upper and a lower matrix is obtained.

The present application finally also relates to a process for the preparation of a sandwich culture of isolated liver cells, where liver tissue of an animal or human body is first prepared and subsequently the liver cells are isolated from the tissue and at least the process steps (a) to (l) according to the invention are carried out. The person skilled in the art will choose, depending on the field of application and suitability, known processes for the isolation of liver cells from tissues.

Finally, the present invention also relates to a sandwich culture which can be prepared by the aforementioned process and is preferably prepared by the process. A sandwich culture according to the invention exhibits all aforementioned advantages and compared to the prior art represents an improved culture of isolated liver cells.

### WORKING EXAMPLES

The invention is explained in more detail in the following examples and figures. The examples are not to be understood as being restrictive, on the contrary, the inventive idea underlying the present invention is thus intended to be explained in more detail and the advantages of the invention illustrated by means of concrete examples.

The figures show:

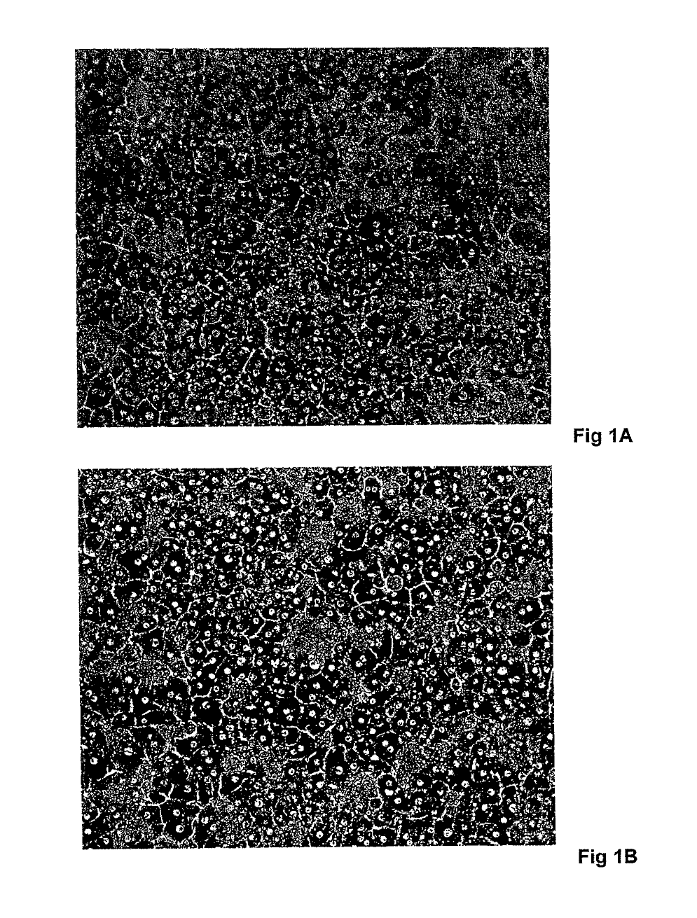

FIG. 1: micrographs of cultured hepatocytes (scale about 150 times); FIG. 1A: freshly isolated human hepatocytes: FIG. 1B: cryopreserved human hepatocytes;

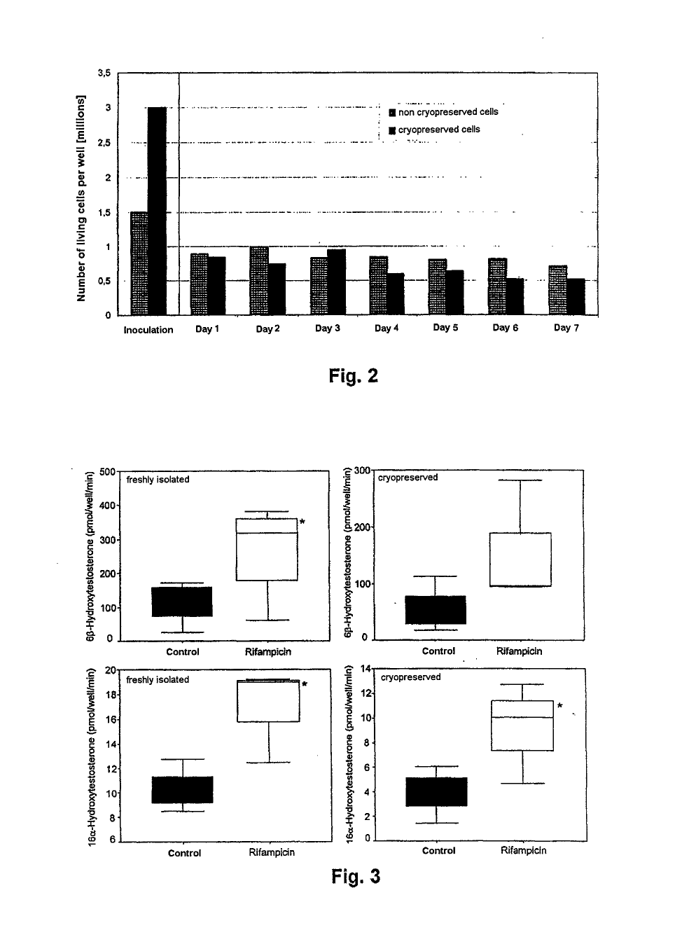

FIG. 2: number of vital hepatocytes as a function of the culturing period;

FIG. 3: formation of 6β-OHT and 16α-OHT after enzyme induction with rifampicin.

### EXAMPLE 1

### Preparation of Human Hepatocytes for Cryopreservation

### 1.1 Isolation of Human Hepatocytes

Hepatocytes from human donors were isolated in a manner known per se from tissue parts anyway removed surgically, which were taken with the agreement of the donor. For this, the tissue was perfused, the hepatocytes detaching from the tissue complex and being able to be obtained from the perfusion solution. The viability of the harvested hepatocytes was determined by means of Trypan Blue assay. For the further experiments, only cell preparations were used which showed more than 70% Trypan Blue exclusion.

### 1.2 Preparation of a Matrix

In the following experiments, cell culture vessels in the form of multiwell plates, 6-well plates (type 657 160, Greiner Bio-One) were used. The plates were coated with native collagen gel, which was preferably isolated from rat tails. Alternatively, multiwell plates, 6-well plates, precoated with collagen type 1 (type 657 950 CELLCOAT, Greiner Bio-One) were used. The 6-well plates had a base area of 9.6 cm² per well.

### 1.3 Inoculation of the Cells

The isolated hepatocytes were inoculated into the multiwell plates. For this, a suspension of the isolated hepatocytes was prepared which contained from 2.5 to 3 million vital hepatocytes per 2 ml of suspension. 2 ml of this cell suspension were pipetted in per well of the 6-well plate. The cell density was thus from 260 to 320 vital hepatocytes per 1 mm² area of the cell culture vessel.

After the inoculation, the plates were allowed to stand for approximately 1 hour. For this, the plates were transferred to a culture cabinet, in which standard conditions (37° C., 5% CO₂, 95% relative humidity) prevailed. The resting phase allowed the cells from the suspension to adhere to the collagen gel. It was possible to assess the successful adhesion by microscopic checking. After the adhesion/resting phase, the supernatant of the cell suspension, which essentially contained nonadherent cells, was aspirated.

1.4 Freezing of the Matrix Coated with Cells

After the aspiration of the supernatant, approximately 1 ml of warm medium 1 at 37° C. was added per well. The cells then rested for approximately one further hour again in the culture cabinet under standard conditions. The supernatant, which essentially contained nonadherent cells, was then thoroughly aspirated.

Medium 1 is derived from a standard cell culture medium for hepatocytes and contains 10% of fetal calf serum (FCS).

For freezing, 0.5 ml of freezing medium per well was added. The addition of the freezing medium was carried out briskly. After addition of the freezing medium, the plates were immediately placed in the freezing machine precooled to 0° C. and the freezing program was configured.

The freezing medium is based essentially on medium 1 and contains 10% of fetal calf serum (FCS) and 10% of dimethyl sulfoxide (DMSO).

The freezing program provided for a compensation of the phase transition and reached a target temperature of −100° C.

The frozen cell culture plates were subsequently stored at −151° C. in a freezer or in the gas phase in a nitrogen tank.

### EXAMPLE 2

### Thawing of Cryopreserved Hepatocytes

The hepatocyte cultures frozen according to Example 1 and stored at −151° C. were thawed and recultured for further use in in vitro tests after a storage period of up to 4 weeks. For the thawing of the cryopreserved hepatocyte cultures, the 6-well plates were first transferred for 5 minutes to a culture cabinet, which was operated using standard conditions, immediately after taking from the freezer or nitrogen tank (see Example 1). Subsequently, 1 ml of medium 1 per well prewarmed to 37° C. (see Example 1) was added slowly and dropwise to each well. This process was repeated for at most three simultaneously thawing 6-well plates, that is for at most 18 wells.

Subsequently, the process was repeated, again in each case 1 ml of medium 1 being slowly added per well. The supernatant was then aspirated with a Pasteur pipette. The supernatant essentially contained thawed nonvital and nonadherent cells.

For further washing, in the same way in each case two times 1 ml of warm medium 2 at 37° C. was added. Here, medium 2, as described above for medium 1, was added to each well in two passages of 1 ml. The plates were then incubated under standard conditions in the culture cabinet for approximately 30 minutes. After the incubation, the complete supernatant, which contained further nonvital and nonadherent cells, was aspirated with a Pasteur pipette.

The monolayer of adherent hepatocytes thus obtained was covered with a further layer of collagen gel, in order to obtain a sandwich configuration. The collagen gel hardened after approximately 30 minutes after pouring on. It had a composition which corresponded essentially to the composition of the lower collagen gel which was introduced into the culture vessels.

Medium 2 is a standard medium for the long-term culturing of hepatocytes and contains 10% of fetal calf serum (FCS).

The further reculturing of the sandwich culture obtained was carried out in medium 2, which was replaced by fresh medium approximately every 24 hours.

### EXAMPLE 3

### Determination of the Number of Viable Cells

The number of viable (vital) cells was determined in cultures of freshly isolated hepatocytes and in cultures of cryopreserved hepatocytes according to the invention by counting the morphologically intact cells. Here, photographs of comparison areas of a size of 0.259 mm² were counted. From the number of the intact cells found in the comparison area, the number of intact cells in the entire cell culture vessel was concluded (for the 6-well plates used having an area of 9.6 cm² per well, a correction factor of 3700 resulted).

Before freezing and at various points in time after thawing after cryopreservation, the morphology of the recultured hepatocytes was documented photographically and compared with cultures of freshly isolated and cultured hepatocytes.

### Results:

FIG. 1 shows the morphology of human hepatocytes which were freshly isolated and inoculated on a collagen gel layer (FIG. 1A) and the morphology of isolated human hepatocytes which were cryopreserved, thawed and recultured for seven days (FIG. 1B). The recultured cryopreserved cells can barely be distinguished morphologically from freshly isolated cells.

The proportion of living cells in the culture was only slightly decreased compared to the proportion of living cells in cultures of freshly isolated hepatocytes. FIG. 2 shows the number of living (vital) human hepatocytes as a function of the reculturing period after thawing the hepatocytes. The comparison curve shows the number of living human hepatocytes on culturing freshly isolated cells over the same period of time. Initially, 3 million cells were inoculated for the cryopreservation, 1.5 million for the fresh preparation.

It is seen that the thawed recultured human hepatocytes grow confluently and that the number of living adherent cells does not differ significantly from the number of living cells in comparable fresh cultures. It is remarkable here that even after a relatively long culturing period the number of living cells in the cryopreserved thawed preparations remains almost constant.

### EXAMPLE 4

### Enzyme Activity of Cryopreserved Hepatocytes

A good marker of the metabolic and/or enzymatic competence of liver cells is the inducibility of the enzymatic hydroxylation of testosterone. In the intact liver cells, a basal level of this reaction exists; hydroxytestosterone (OHT) is formed here. The formation rates can be increased in intact cells by enzyme induction. The detection of the formation of OHT in cultured hepatocytes can therefore allow conclusions on the enzymatic competence and a physiological function of the cultured hepatocytes.

Analysis and quantification of the regio- and/or stereo-selective testosterone hydroxylation was carried out in a manner known per se, published, for example, in Friedrich et al., 2003 (J. Chromatogr. B. 78 4:49-61).

For this investigation, human hepatocytes cryopreserved according to the invention were thawed and recultured for two days and subsequently incubated in rifampicin for a further 24 hours. Rifampicin induces testosterone hydroxylation in the positions 6β, 16α and 2β. The testosterone hydroxylation in position 6α is not stimulated by rifampicin. The measurement of 6α-hydroxylated testosterone therefore served for the comparison measurement of the enzyme induction by rifampicin.

As a control, freshly isolated cultures were likewise incubated in rifampicin for 24 hours after they had been cultured for two days. The culturing conditions were chosen analogously to the process according to the invention.

In addition to the enzyme inducibility, the testosterone basal level was determined in the cryopreserved and recultured cells and in the freshly prepared and cultured cells.

### Results:

Cryopreserved, recultured hepatocytes according to the invention and freshly prepared cultured hepatocytes showed comparable enzyme inducibility. The mean values for the induction of testosterone hydroxylation are shown in the following table:

| | Cryopreserved, | |
|---|---|---|
| | recultured | Freshly isolated |
| | hepatocytes (acc. | hepatocytes |
| | to the invention) | (comparison example) |
| 6β-hydroxylation | 2.8-fold | 2.3-fold |
| 16α-hydroxylation | 2.4-fold | 1.6-fold |
| 2β-hydroxylation | 2.3-fold | 2.4-fold |

As expected, rifampicin was unable to induce the formation of 6α-hydroxytestosterone (6α-OHT) either in the cryopreserved and recultured hepatocytes or in the freshly cultured hepatocytes.

FIG. 3 shows the absolute concentration of hydroxy-testosterone formed (shown by way of example for 6β-OHT and 16α-OHT) before and after induction of testosterone hydroxylation with rifampicin. In the left part of the figure the results for the freshly cultured hepatocytes are shown, in the right part of the figure the results for the cryopreserved and recultured hepatocytes according to the invention. FIG. 3A shows the formation of 6β-OHT, FIG. 3B shows the formation of 16α(—OHT. The box and whisker blots indicate the quartiles. The significance level indicates p <0.05 (t test).

From experience with liver cells cryopreserved in suspension, it is known that cryopreservation greatly decreases the basal level during testosterone hydroxylation compared to freshly prepared hepatocytes. As expected, the basal activity of the recultured hepatocytes also decreased in the liver cells cryopreserved in the monolayer in comparison to the fresh culture. It was also still detectable, however, 24 and 72 hours after thawing.

It is particularly seen that the cryopreserved hepatocytes exhibit a marked inducibility of testosterone hydroxylation at a point in time 72 hours after thawing and after prior 24-hour incubation with rifampicin.

The basal activity obtained over at least three days together with the undecreased inducibility are essential indications of the fact that the cryopreserved human hepatocytes according to the invention can be employed successfully for a large number of in vitro tests.

1. Process for the preparation of liver cells for cryopreservation, comprising the steps:
(a) preparing a matrix,
(b) preparing isolated liver cells,
(c) inoculating the liver cells on the matrix in a density of 2 to 4×10³ mm⁻²,
(d) allowing the matrix coated with cells to rest for 10 to 180 min, so that cells can adhere to the matrix,
(e) washing off nonadherent cells from the matrix coated with cells,
(f) allowing the matrix coated with cells to rest for up to at most 180 min, and
(g) freezing the matrix coated with cells in a freezing medium.

2. The process as claimed in claim 1, wherein the matrix is a collagen matrix.

3. The process as claimed in claim 1, wherein the resting in step (d) takes place for 30 to 90 min.

4. The process as claimed in claim 1, further comprising covering in step (e) the matrix coated with cells with a layer of culture medium and wherein, in a further step, liquid supernatant above the matrix coated with cells is subsequently aspirated.

5. The process as claimed in claim 1, wherein the resting in step (f) takes place for 30 to 180 min.

6. The process as claimed in claim 1, wherein freezing medium in an amount of 0.5 μl mm⁻² is added in step (g).

7. The process as claimed in claim 6, wherein the freezing medium comprises fetal calf serum (FCS) and DMSO.

8. The process as claimed in claim 1, wherein the freezing step (g) comprises covering the matrix coated with cells with freezing medium and controlling cooling to −80° C. or less at cooling rates of 0.5 to 20° C. min⁻¹.

9. A process for the storage and reculturing of isolated liver cells, comprising the steps:
(a) to (g) as claimed in claim 1,
(h) storing the frozen matrix coated with cells,
(i) thawing the frozen matrix coated with cells,
(j) washing off nonadherent cells from the matrix coated with cells,
(k) coating the thawed matrix coated with cells with a layer of a second matrix, and
(l) reculturing the cells embedded between the matrices.

10. The process as claimed in claim 9, wherein the storage in step (h) takes place at −150° C.

11. The process as claimed in claim 9, wherein the matrix coated with cells is covered with a layer of warm culture medium in step (i).

12. The process as claimed in claim 9, wherein the matrix coated with cells is covered with a layer of culture medium in step (j) and liquid supernatant above the matrix coated with cells is subsequently aspirated.

13. A process for the preparation of a sandwich culture of isolated liver cells, comprising the steps (a) to (l) of the process as claimed in claim 9.

14. The process as claimed in claim 13, wherein step (a) comprises the steps:
(a1) preparing liver tissue from an animal or human body and
(a2) isolating liver cells from the tissue.

15. A sandwich culture of liver cells, which can be prepared by the process of claim 13.

16. The process as claimed in claim 2, wherein the resting in step (d) takes place for 30 to 90 min.

17. The process as claimed in claim 4, wherein the freezing step (g) comprises covering the matrix coated with cells with freezing medium and controlling cooling to −80° C. or less at cooling rates of 0.5 to 20° C. min⁻¹.

18. The process as claimed in claim 8, further including compensation of phase transition.

19. The process as claimed in claim 10, wherein the matrix coated with cells is covered with a layer of warm culture medium in step (i).

20. A sandwich culture of liver cells, prepared by the process of claim 14.
